(19) United States
(12) Patent Application Publication
(10) Pub. No.: US 2006/0069348 A1
(43) Pub. Date: Mar. 30, 2006

(54) Hypodermic Syringes

(76) Inventors: David William Parker, Bury (GB); Colin H Burgess, Ramsbottom (GB)

Correspondence Address: NIXON & VANDERHYE, PC, 901 NORTH GLEBE ROAD, 11TH FLOOR, ARLINGTON, VA, 22203, US

(21) Appl. No.: 10/529,722

(22) PCT Filed: Oct. 1, 2003
(86) PCT No.: PCT/GB03/04326

§ 371 Date: Jun. 24, 2005

(30) Foreign Application Priority Data
Oct. 1, 2002 (GB) 0222731.2

(51) Int. Cl. A61M 5/00; A61M 5/315; A61M 5/31

(52) U.S. Cl. 604/110; 604/218; 604/243

(57) ABSTRACT

A hypodermic syringe comprises a housing (1), which includes a barrel portion (1.4) and an injectant chamber (1.5), where the injectant chamber has a smaller cross-sectional area than the barrel portion (1.4). A plunger (2), which comprises a piston (3), is slideably mounted within the barrel portion (1.4) and extends into the injectant chamber (1.5). A retractable needle assembly (5) is provided, together with stored energy means (4), so that, at the completion of an injection stroke, the piston (3) becomes attached to the needle assembly (5) and the stored energy is released to retract the needle assembly (5) into the housing (1). Co-operating features (3.7, 5.7) may be arranged on the piston (3) and needle assembly (5) so that complete evacuation of the injectant chamber (1.5) is not impeded. As the parameters of the stored energy means (4) are not limited by the physical dimensions of the injectant chamber (1.5), this arrangement is particularly suitable for small capacity syringes, i.e. 3 ml or less.

This invention relates to hypodermic syringes where a needle assembly may be retracted into a syringe housing after use. In particular, the invention relates to a retractable hypodermic syringe suitable for the delivery of small doses of injectant.

It is now well-known that there is a worldwide need for safe hypodermic syringes, the most effective of these being a passive syringe with a retracting needle. Examples of such syringes are disclosed in our co-pending patent applications WO00/18454, WO01/43619 and WO01/72362.

However, the provision of a needle retraction mechanism in a small capacity syringe is not straightforward. The reliability of a needle retraction mechanism can only be ensured if adequate stored energy is provided to overcome friction between components of the syringe and any resistance from a bent needle. Prior syringes have comprised stored energy configurations that are limited by the dimensions of the syringe, i.e. based on the cross-sectional area of the injectant chamber. The dimensions of the injectant chamber of a small capacity syringe typically range from around 6 mm in diameter, giving a cross sectional area of 28 mm², for a 1 ml. syringe to 10 mm diameter, i.e. a cross sectional area of 78 mm², for a 3 ml. syringe. making the provision of a suitable stored energy means problematical.

An object of the present invention is to provide a retraction facility that is particularly suitable for syringes of small capacity, i.e. 3 ml or less.

In a first aspect of the present invention, a hypodermic syringe comprises a housing, said housing including a barrel portion and an injectant chamber, the injectant chamber being of a smaller cross-sectional area than the barrel portion, a plunger slideably mounted within the barrel portion, comprising a piston which extends into the injectant chamber, a retractable needle assembly and a stored energy means, configured so that, at the completion of an injection stroke, the piston may become attached to the needle assembly and the stored energy in the stored energy means released to retract the needle assembly into the housing.

In a second aspect of the present invention, a small capacity hypodermic syringe comprises a housing including an injectant chamber of small cross-sectional area, a retractable needle assembly, a stored energy means for effecting retraction and a plunger, wherein the parameters of a stored energy means are not limited by the dimensions of the injectant chamber.

In a third aspect of the present invention, a hypodermic syringe comprises, a housing including an injectant chamber, a plunger slideably mounted within the housing, a piston mounted on the plunger comprising a first co-operating feature, a retractable needle assembly comprising a second co-operating feature and a stored energy means for effecting the retraction of the needle assembly, wherein the first and second co-operating features are configured to lock together at the completion of an injection stroke, said the co-operating features being arranged so as not to impede the complete evacuation of the injectant chamber.

The invention will now be described by way of an example with reference to the accompanying drawing.

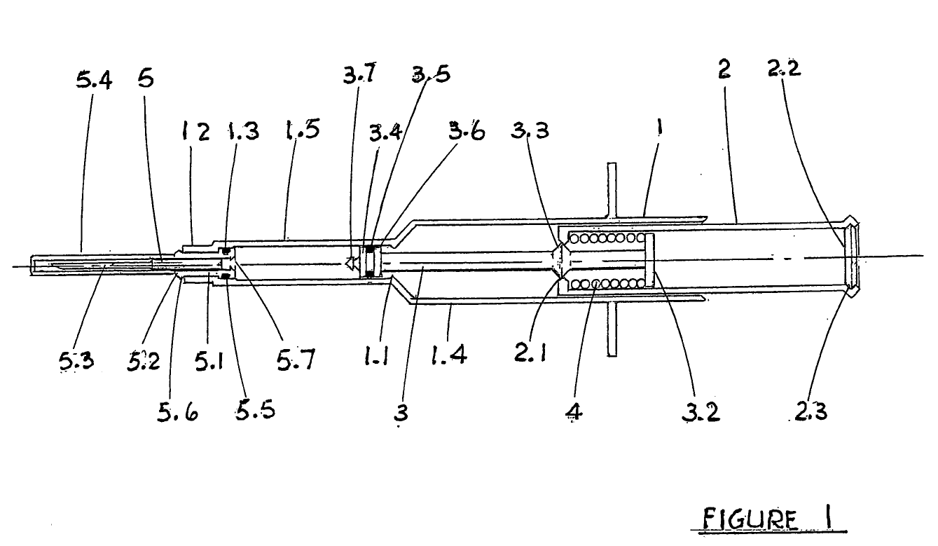

FIG. 1 depicts a syringe according to the present invention, comprising a body 1 with a proximal end and a distal end 1.2, including a barrel 1.4, a constriction 1.1, a portion of reduced diameter 1.3 and an injectant chamber 1.5. An assembly including a plunger 2, a piston rod 3 and a spring 4 is slideably mounted within the body 1.

The body 1 houses a needle assembly 5, which comprises of a two part needle carrier 5.1, 5.2 and a seal 5.5 and is mounted in the body 1 by a snap-fit. A sheath 5.4 is also provided, which covers a needle 5.3 mounted on the needle assembly 5. The first part 5.1 of the needle assembly 5 has a non-circular cross-section and is located in a non-circular aperture in the distal end 1.2 of the body 1, in order to prevent rotation of the needle 5.3 relative to the body 1. This facilitates subsequent removal of the sheath 5.4 so that the sheath 5.4 can be removed using a twisting motion, e.g. by gripping the sheath 5.4 and rotating the body 1 without causing such a rotation.

The needle assembly 5, needle 5.3 and sheath 5.4 are dimensioned to pass through the constriction 1.1 in the body 1. This allows positioning of the needle assembly 5 within the body 1 during assembly of the syringe with the needle 5.3 pre-sheathed. The needle assembly 5, needle 5.3 and sheath 5.4 are inserted through the proximal end of the body 1 and snap into a position in the distal end 1.2 of the body 1. In this position, the seal 5.5 is located in a reduced diameter portion 1.3 of the injectant chamber 1.5. The needle carrier part 5.2 includes a feature 5.6, e.g. a button or ridge, which provides a reversible retaining catch against the body 1.

The piston 3 is fitted with the spring 4 bearing against a flange 3.2 and is mounted through the proximal end of the plunger 2. The spring 4 is compressed until a reversible catch formed by a collar 3.3 and an orifice 2.1 is engaged to retain the spring 4 under compression.

The assembly thus formed of plunger 2 and piston 3 is inserted into the barrel 1.4 of the body 1. When the piston 3 meets the constriction 1.1, a flange 3.4 and a seal 3.5 located on the piston 3 will pass through with little resistance, but a second flange 3.6 is so dimensioned that it snaps through the restriction 1.1, with which it interacts to form a virtually non-reversible catch. A plunger closure piece 2.2, which may have a central hole for assembly purposes, is snap-fitted into a recess 2.3 formed at the proximal end of the plunger 2.

Operation of the syringe follows closely established practice. Following removal of the sheath 5.4, the plunger 2 is pressed in order to cause air in the injectant chamber 1.5 to be expelled, but not so far as to engage a non-reversible catch formed by a socket 5.7 located in the needle assembly 5 and a barb 3.7 on the piston 3. The injectant is drawn in the usual way and any necessary adjustments are made to expel air and to ensure that the correct quantity of injectant has been loaded.

Pressure is applied to the plunger 2 in the usual way to effect injection, expelling the injectant. At the end of an injection stroke, an end face of the piston 3 abuts the needle carrier 5 and the two interacting features, i.e. the socket 5.7 and the barb 3.7 engage.

Further pressure on the plunger 2 will cause reversal of the catch formed by the collar 3.3 of the piston 3 and the orifice 2.1 of the plunger 2, releasing the stored energy of the spring 4 so that the needle assembly 5 is retracted. The design ensures that a spring 4 of sufficient strength can be used to overcome the friction of the piston seal 3.5 and the reversible catch formed by the feature 5.6 against the distal end of the body 1. Once released, the needle assembly 5 and needle 5.3, both being of lesser diameter than the injectant chamber 1.5, do not resist retraction.

The needle retraction movement is halted by the piston flange 3.6 meeting the constriction 1.1 in the body 1. The remaining spring energy draws the plunger 2 into the body 1 so that the plunger closure piece 2.2 is flush with the proximal end of the body 1 and the whole assembly is locked in a secure position.

In addition to providing a reliable needle retraction mechanism, the embodiment also has the following attributes:
Accurate metering;
   In order to achieve this, good visibility of the piston 3 and injectant chamber 1.5 is maintained, as the components of the needle retraction mechanism do not obscure the user's view of the injectant chamber 1.5. Furthermore, as the injectant chamber 1.5 has a small cross-section, the length of the injectant chamber 1.5 is sufficient to allow cleat calibration spacing;
Complete evacuation of the injectant chamber;
   Complete evacuation is desirable for reasons of injectant cost or aggressiveness. This attribute is provided by maximising the contact between a needle piston 3 and the body 1, in particular the contact between the piston flange 3.4 and the distal end 1.2 of the body. This allows the piston 3 to sweep the total length of the injectant chamber 1.5 so that the evacuation of the injectant is not impeded by any components of the needle release mechanism;
Security after use for safe disposal;
   The needle is locked in a retracted position after use without requiring any additional action by the user, i.e. in a completely automatic process forming part of the needle retraction sequence;
   Instinctive operation following established principles;
   The syringe maintains the feel of a standard syringe in both handling and operation, thereby reducing both the incidence of errors and the need for special training of its users.
Low cost and simple assembly;
   Low unit cost is achieved as the syringe contains a reduced the number of components, when compared with standard retractable syringes. In addition, the components are of forms that can be simply moulded. The simple assembly process described above is also a significant contributor to low cost.

The embodiment described comprises a needle which is fitted during the manufacturing process, as is usual for syringes of small capacity. However, if a need arises for needle replacement, or for the fitting a needle to the syringe by the user, the syringe may allow the threading of part of a needle between the first and second parts 5.1, 5.2 of the needle assembly. Similarly, while the injectant chamber 1.5 of the embodiment is cylindrical, i.e. with a circular cross-section, it is not necessary for the injectant chamber to have this particular shape.

1. A hypodermic syringe comprising: a housing, said housing including a barrel portion and an injectant chamber, the injectant chamber having a smaller cross-sectional area than the barrel portion; a plunger slideably mounted within the barrel portion, comprising a piston which extends into the injectant chamber; a retractable needle assembly; and a stored energy means; configured so that, at the completion of an injection stroke, the piston may become attached to the needle assembly and the stored energy in the stored energy means released to retract the needle assembly into the housing.

2. A hypodermic syringe according to claim 1, wherein the stored energy means is located in the plunger.

3. A hypodermic syringe according to claim 1, wherein, following needle retraction, residual stored energy is used to retain the needle and plunger within the housing.

4. A hypodermic syringe according to claim 1, further comprising non-reversible snap-fitting formations for securely retaining the needle assembly within the syringe after use.

5. A hypodermic syringe according to claim 1, wherein a first part of the needle assembly has a non-circular cross-section and is located in a non-circular aperture of the body, wherein the first part and the aperture co-operate to prevent rotation of the needle assembly relative to the body.

6. A hypodermic syringe according to claim 1, wherein the injectant chamber has a capacity of 3 ML or less.

7. A small capacity hypodermic syringe comprising a housing including an injectant chamber of small cross-sectional area, a retractable needle assembly, a stored energy means for effecting retraction and a plunger, wherein the parameters of a stored energy means are not limited by the dimensions of the injectant chamber.

8. A hypodermic syringe according to claim 7 in which the stored energy means is a spring.

9. A hypodermic syringe according to claim 7, wherein during assembly of the syringe, the needle assembly is mounted in the housing with the needle pre-sheathed.

10. A hypodermic syringe according to claim 7, wherein the cross-sectional area of the needle assembly is smaller than the cross-sectional area of the injectant chamber and the needle assembly comprises a seal in order to reduce friction during needle retraction.

11. A hypodermic syringe according to claim 7 wherein, following needle retraction, residual spring energy is used to retain the needle and plunger within the housing.

12. A hypodermic syringe according to claim 7, further comprising non-reversible snap-fitting formations for securely retaining the needle assembly within the syringe after use.

13. A hypodermic syringe according to claim 1 in which one or more components of the syringe are located and retained by the use of snap-fits during assembly.

14. A hypodermic syringe according to claim 1, further comprising a plunger closure piece, said plunger closure piece having an aperture.

15. A hypodermic syringe comprising: a housing, said housing including an injectant chamber; a plunger slideably mounted within the housing; a piston mounted on the plunger comprising a first CO-OPERATING feature; a retractable needle assembly comprising a second co-operating feature; and a stored energy means for effecting the retraction of the needle assembly, wherein the first and second CO-OPERATING features are configured to lock together at the completion of an injection stroke, said the co-operating features being arranged so as not to impede the complete evacuation of the injectant chamber.

16. A hypodermic syringe according to claim 15, wherein the injectant chamber has a capacity equal to or less than 3 ML.
